(19) United States
(12) Patent Application Publication
(10) Pub. No.: US 2016/0097085 A1
(43) Pub. Date: Apr. 7, 2016

(54) Compositions and Methods for cDNA Synthesis

(71) Applicant: Jun Euihum Lee, San Diego, CA (US)

(72) Inventor: Jun Euihum Lee, San Diego, CA (US)

(21) Appl. No.: 14/506,568

(22) Filed: Oct. 3, 2014

(51) Int. Cl. C12Q 1/68

(52) U.S. Cl. CPC C12Q 1/686; C12Q 1/6851

(57) ABSTRACT

The methods and compositions for preparing cDNAs, and more particularly, compositions having propylene glycol for synthesizing a cDNA molecule or molecules from an mRNA template or population of mRNA templates under conditions sufficient to increase the detection sensitivity and cDNA yield and to simplify and improve the reliability of reverse transcription are provided. The reagent mixture comprises a ready to use reagent solution, wherein the solution comprises: (a) propylene glycol in a concentration between about 25% and about 50%; and (b) a viral reverse transcriptase selected from the group consisting of AMV RT, RSV RT, MMLV RT, HIV RT, EIAV RT, RAV2 RT, ASLV RT, RNaseH (−) RT, SuperScript II RT, and ThermoScript RT, in a buffer suitable for use in a reverse transcription reaction, wherein the buffer further comprises a co-factor metal ion and nucleoside triphosphates.

### FIELD OF THE INVENTION

The present invention relates to compositions and methods for preparing complementary deoxyribonucleic acids (cDNAs), and more particularly, compositions having propylene glycol for synthesizing a cDNA molecule or cDNA molecules from a messenger ribonucleic acid (mRNA) template or population of mRNA templates under conditions sufficient to increase the detection sensitivity and cDNA yield and to simplify and improve the reliability of reverse transcription.

### BACKGROUND OF THE INVENTION

Within a given cell, tissue or organism, there exist many mRNA species, each encoding a separate and specific protein. The identity and levels of specific mRNAs present in a particular sample provide clues to the biology of the particular tissue or sample being studied. Therefore, the detection, analysis, transcription, and amplification of RNAs are one of the most important procedures in modern molecular biology.

A common approach to the study of gene expression is the production of cDNA. In this technique, the mRNA molecules are isolated from the extraction of cells or tissues of an organism. From these purified mRNA molecules, cDNA copies may be made using the enzyme reverse transcriptase (RT), which results in the production of single-stranded cDNA molecules.

Avian myoblastosis virus (AMV) reverse transcriptase was the first widely used RNA dependent DNA polymerase (Verma, Biochem. Biophys. Acta 473:1(1977)). The enzyme has 5′-3′ RNA directed DNA polymerase activity, 5′-3′ DNA directed DNA polymerase activity, and RNase H activity. RNase H is a processive 5′ and 3′ ribonuclease specific for the RNA strand for RNA-DNA hybrids (Perbal, A Practical Guide to Molecular Cloning, New York: Wiley & Sons (1984)). A detailed study of the activity of AMV reverse transcriptase and its associated RNase H activity has been presented by Berger, et al., Biochemistry 22:2365 2372 (1983). Another reverse transcriptase which is used extensively in molecular biology is reverse transcriptase originating from Moloney murine leukemia virus (M-MLV). See, e.g., Gerard, G. R., DNA 5:271 279 (1986) and Kotewicz, M. L., et al., Gene 35:249 258 (1985). M-MLV reverse transcriptase substantially lacking in RNase H activity has also been described. See, e.g., U.S. Pat. No. 5,244,797.

One of the most widely used techniques to study gene expression exploits first-strand cDNA for mRNA sequence(s) as template for amplification by the polymerase chain reaction (PCR). This method, often referred to as RNA PCR or reverse transcriptase PCR (RT-PCR), exploits the high sensitivity and specificity of the PCR process and is widely used for detection and quantification of RNA. Recently, the ability to measure the kinetics of a PCR reaction by on-line detection in combination with these RT-PCR techniques has enabled accurate and precise measurement of RNA sequences with high sensitivity. This has become possible by detecting the RT-PCR product through fluorescence monitoring and measurement of PCR product during the amplification process by fluorescent dual-labeled hybridization probe technologies, such as the “TaqMan” 5′ fluorogenic nuclease assay described by Holland, at al. (Proc. Natl. Acad. Sci. U.S.A. 88, 7276 (1991)), and Gibson, et al. (Genome Res. 6, 99 (1996) or “Molecular Beacons” (Tyagi, S. and Kramer, F. R. Nature Biotechnology 14, 303 (1996)) has described use of dual-labeled hairpin primers. One of the more widely used methods is the addition of double-strand DNA-specific fluorescent dyes to the reaction such as SYBR Green I (Wittwer, at al., Biotechniques 22,130 (1997). These improvements in the PCR method have enabled simultaneous amplification and homogeneous detection of the amplified nucleic acid without purification of PCR product or separation by gel electrophoresis. This combined approach decreases sample handling, saves time, and greatly reduces the risk of product contamination for subsequent reactions, as there is no need to remove the samples from their closed containers for further analysis. The concept of combining amplification with product analysis has become known as “real time” PCR, also referred to as quantitative PCR, or qPCR. The general principals for template quantification by real-time PCR were first disclosed by Higuchi R, G Dollinger, P S Walsh and R. Griffith. Use of real time PCR methods provides a significant improvement towards this goal. However, real-time PCR quantification of mRNA is still bounded by limitations of the process of reverse transcription.

The RT-PCR procedure, carried out as either an end-point or real-time assay, involves two separate molecular syntheses: (i) the synthesis of cDNA from an RNA template; and (ii) the replication of the newly synthesized cDNA through PCR amplification. To attempt to address the technical problems often associated with RT-PCR, a number of protocols have been developed taking into account the three basic steps of the procedure: (a) the denaturation of RNA and the hybridization of reverse primer; (b) the synthesis of cDNA; and (c) PCR amplification. In the so called “uncoupled” RT-PCR procedure (e.g., two step RT-PCR), reverse transcription is performed as an independent step using the optimal buffer condition for reverse transcriptase activity. Following cDNA synthesis, the reaction is diluted to decrease MgCl₂, and deoxyribonucleoside triphosphate (dNTP) concentrations to conditions optimal for Taq DNA Polymerase activity, and PCR is carried out according to standard conditions (see U.S. Pat. Nos. 4,683,195 and 4,683,202). By contrast, “coupled” RT PCR methods use a common or compromised buffer for reverse transcriptase and Taq DNA Polymerase activities. In one version, the annealing of reverse primer is a separate step preceding the addition of enzymes, which are then added to the single reaction vessel. In another version, the reverse transcriptase activity is a component of the thermostable Tth DNA polymerase. Annealing and cDNA synthesis are performed in the presence of Mn⁺⁺ then PCR is carried out in the presence of Mg⁺⁺ after the removal of Mn⁺⁺ by a chelating agent. Finally, the “continuous” method (e.g., one step RT-PCR) integrates the three RT-PCR steps into a single continuous reaction that avoids the opening of the reaction tube for component or enzyme addition.

One step RT-PCR provides several advantages over uncoupled RT-PCR. One step RT-PCR requires less handling of the reaction mixture reagents and nucleic acid products than uncoupled RT-PCR (e.g., opening of the reaction tube for component or enzyme addition in between the two reaction steps), and is therefore less labor intensive, reducing the required work hours. One step RT-PCR also requires less sample, and reduces the risk of contamination (Sellner and Turbett, 1998). The sensitivity and specificity of one-step RT-PCR has proven well suited for studying expression levels of one to several genes in a given sample or the detection of pathogen RNA. Typically, this procedure has been limited to use of gene-specific primers to initiate cDNA synthesis.

In contrast, use of non-specific primer in the “uncoupled” RT-PCR procedure provides opportunity to capture all RNA sequences in a sample into first-strand cDNA, thus enabling the profiling and quantitative measurement of many different sequences in a sample, each by a separate PCR. The ability to increase the total amount of cDNA produced, and more particularly to produce cDNA that truly represents the mRNA population of the sample would provide a significant advance in study of gene expression. Specifically, such advances would greatly improve the probability of identifying genes which are responsible for disease in various tissues.

Ideally, synthesis of a cDNA molecule initiates at or near the 3′-termini of the mRNA molecules and terminates at the mRNA 5′-end, thereby generating “full-length” cDNA. Priming of cDNA synthesis at the 3′-termini at the poly A tail using an oligo deoxy-thymine (dT) primer ensures that the 3′-message of the mRNAs will be represented in the cDNA molecules produced. It would be very desirable if cDNA synthesis initiated at 3′ end and continued to the 5′-end of mRNA's regardless of length of mRNA and the reverse transcriptase used. However, due to many factors such as length, nucleotide sequence composition, secondary structure of mRNA and also inadequate processivity of reverse transcriptases, cDNA synthesis prematurely terminates resulting in non-quantitative representation of different regions of mRNA (i.e. 3′-end sequences or 5′-end sequences). It has been demonstrated that use of mutant reverse transcriptases lacking RNase H activity result in longer cDNA synthesis and better representation, and higher sensitivity of detection. However, it is generally believed that using oligo dT primer results in cDNA sequence bias of mRNA 3′-end region.

In studies involving quantitative analysis of gene expression, sequence bias in the cDNA and non-quantitative representation of different parts of mRNA can yield inaccurate expression data. Due to these problems an alternative method of priming for cDNA synthesis has been used utilizing random primers. Due to random sequence, these primers are believed to non-specifically prime cDNA synthesis at arbitrary sites along the mRNA resulting shorter cDNA fragments that collectively represent all parts of mRNA in the cDNA population. Gerard and D'Alessio (1993 Methods in Molecular Biology 16:73-93) have reported that the ratio of random primer to mRNA is critical for efficient cDNA synthesis by M-MLV RT or its RNase H deficient derivatives. Increasing concentrations of random hexamer resulted in increased yields of cDNA, however the average length of cDNA decreased accordingly. This indicates that primer concentration must be optimized for different amounts of starting RNA template to achieve efficient cDNA synthesis efficiency. Since random primer has the potential to omit sequence close to the mRNA polyA tail, in some protocols, oligo dT primer and random primers have been used as mixtures and combine both priming methods.

The choice and concentration of primer can have a profound impact on the quantitative representation of different mRNA transcripts in first-strand cDNA. It is apparent therefore, that improved compositions and methods for improving the yield of cDNA produced using reverse transcription are greatly desired. It is also apparent that new methods for making collections or libraries of cDNA from cells or tissue that more accurately represent the relative amounts of mRNAs present in the cells or tissue are greatly desired. It is also apparent that more convenient compositions and kits for use in such methods are desirable.

### SUMMARY OF THE INVENTION

The present invention provides methods for making cDNA molecules, for amplification of cDNA by PCR.

The present invention also provides kits for making non-biased cDNA molecules. Convenient and ready-to-use cDNA synthesis mastermix compositions are also provided. The comprising mixtures of reagents include reverse transcriptases, buffers, cofactors and other components, suitable for immediate use in conversion of RNA into cDNA and following amplification of cDNA by PCR. These mastermix compositions are useful, alone or in the form of kits, for cDNA synthesis or nucleic acid amplification (e.g., PCR) or for any procedure utilizing reverse transcriptases in a variety of research, medical, diagnostic, forensic and agricultural applications.

The present invention is directed to compositions comprising mixtures of reagents, including reverse transcriptases, buffers, cofactors and other components, suitable for immediate use in conversion of RNA into cDNA and RT PCR without dilution or addition of further components. These compositions are useful, alone or in the form of kits, for cDNA synthesis or nucleic acid amplification (e.g., PCR) or for any procedure utilizing reverse transcriptases in a variety of research, medical, diagnostic, forensic and agricultural applications.

The invention also provides improved methods of synthesizing a cDNA molecule(s) from an mRNA templates under conditions sufficient to increase the detection sensitivity and cDNA yield. Specifically, the invention relates to the use of a mixture of oligo(dT) primer and random primer in a first-strand cDNA synthesis reaction.

In one aspect of the invention, the buffer may comprise a monovalent cation selected from the group consisting of Na, K, and NH₄, a magnesium salt, a reducing agent, nucleoside triphosphates, and at least one non-ionic detergent. The buffer may further comprise at least one primer suitable for priming reverse transcription of a template by the reverse transcriptase. The mixture may also comprise an RNase inhibitor protein.

In one embodiment, the buffer comprises a potassium salt, a magnesium salt, nucleoside triphosphates, DTT, at least one primer suitable for priming reverse transcription of a template by the reverse transcriptase, at least one non-ionic detergent, and an RNase inhibitor protein.

In any of these methods and compositions, two or more reverse transcriptases may be used, including any reverse transcriptase as described above.

Although the present invention is briefly summarized, the fuller understanding of the invention can be obtained by the following drawings, detailed description and appended claims. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

These and other features, aspects and advantages of the present invention will become better understood with reference to the accompanying drawings, wherein:

FIG. 1 shows the freeze-thaw stability information of various cDNA synthesis mastermixes according to the number of freeze-thaw cycles Master Mix has gone through;

FIG. 2 shows performance of various concentrations of propylene glycol mastermixes;

FIG. 3 shows the batch to batch reproducibility and reliability of the cDNA synthesis mastemixes; and

FIG. 4 shows the performances of the cDNA Synthesis Master Mix on five different mRNA targets.

### DETAILED DESCRIPTION OF THE INVENTION

The present invention relates to methods of increasing the efficiency of cDNA synthesis and more particularly, to increasing the sensitivity and accuracy of quantification of gene expression. Thus, the present invention provides improved cDNA synthesis useful in gene discovery, genomic research, diagnostics and identification of differentially expressed genes and identification of genes of importance to disease.

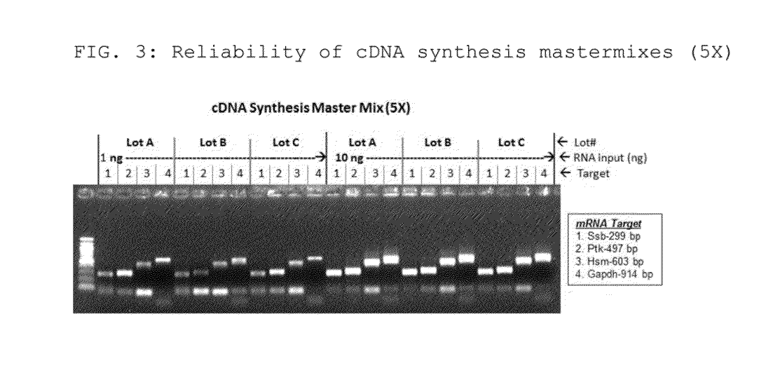

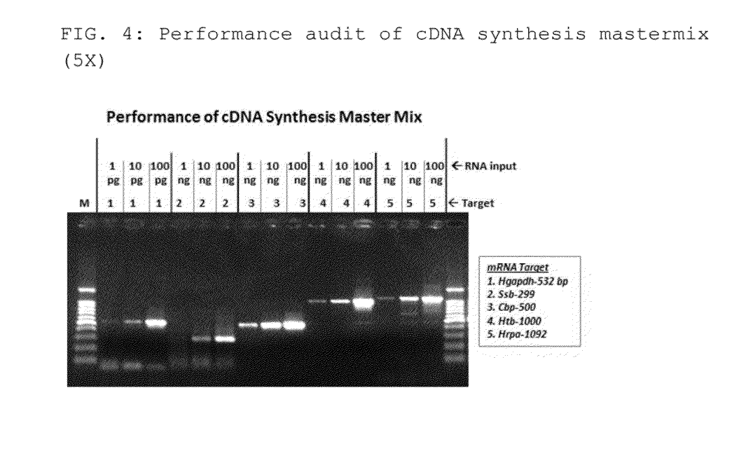

Other embodiments of the invention relate to stabilized concentrated reaction mixtures for first-strand cDNA synthesis that simplify and improve the reliability of reverse transcription (See FIGS. 3 and 4).

The reagent mixture of the present invention comprises a ready to use reagent solution that demonstrates prolonged stability when stored at −20° C. The solution comprises (a) Propylene glycol in a concentration between about 25% and about 50%; and (b) a viral reverse transcriptase in a concentration sufficient for use in a reverse transcription reaction without adding additional reverse transcriptase, wherein the viral reverse is selected from the group consisting of Avian Myeloblastosis Virus Reverse Transcriptase (AMV RT), Respiratory Syncytial Virus Reverse Transcriptase (RSV RT), Moloney Murine Leukemia Virus Reverse Transcriptase (MMLV RT), Human Immunodeficiency Virus Reverse Transcriptase (HIV RT), Equine Infectious Anemia Virus Reverse Transcriptase (EIAV RT), Rous-Associated Virus 2 Reverse Transcriptase (RAV2RT), Avian Sarcoma Leukosis Virus Reverse Transcriptase (ASLVRT), RNaseH (−) Reverse Transcriptase, SuperScript II Reverse Transcriptase, and ThermoScript Reverse Transcriptase, in a buffer suitable for use in a reverse transcription reaction. The buffer comprises a co-factor metal ion necessary for reverse transcriptase activity and nucleoside triphosphates.

The buffer may further comprise at least one primer suitable for priming reverse transcription of a template by the reverse transcriptase. The buffer may comprise an RNase inhibitor protein. In addition, the buffer may comprise a potassium salt, a magnesium salt, nucleoside triphosphates, Dithiothreitol, at least one primer suitable for priming reverse transcription of a template by said reverse transcriptase, at least one non-ionic detergent, and an RNase inhibitor protein.

The solution may comprise one or two or more viral reverse transcriptase enzymes. The buffer may comprise at least one random primer and the random primer may be hexameric primer, heptameric primer, octameric primer, etc. (0.2 ug/ml-5 ug/ml).

The buffer may comprise at least one oligo deoxy-thymine primer such as deoxy-thymine 6-10 or deoxy-thymine 12-18. In addition, the buffer may comprise at least one random primer and at least one oligo deoxy-thymine primer.

The metal ion necessary for reverse transcriptase activity may be magnesium ion preferably in the amount of 2-20 mM. In addition, the buffer may further comprise a monovalent cation such as K, Na, or NH₄ in the amount of 20-500 mM.

The buffer may comprise a reducing agent and the reducing agent may be Dithiothreitol in the amount of 2-50 mM. The buffer may further comprise a non-ionic detergent (e.g., NP-40, Tween-20, 0.001-0.1%), trehalose (30-300 mM), and/or glycerol (2-9%).

The solution is stable for 12 months to 2 years when stored at about −20 degree. The solution shows better performances when the propylene glycol is in a concentration between about 30% and about 40%.

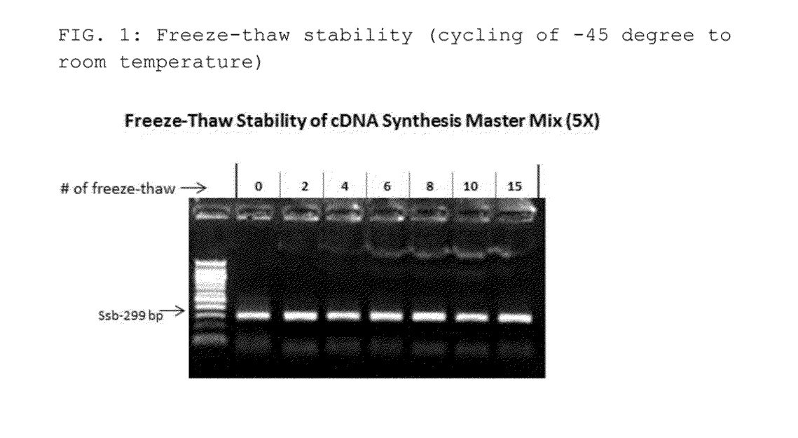

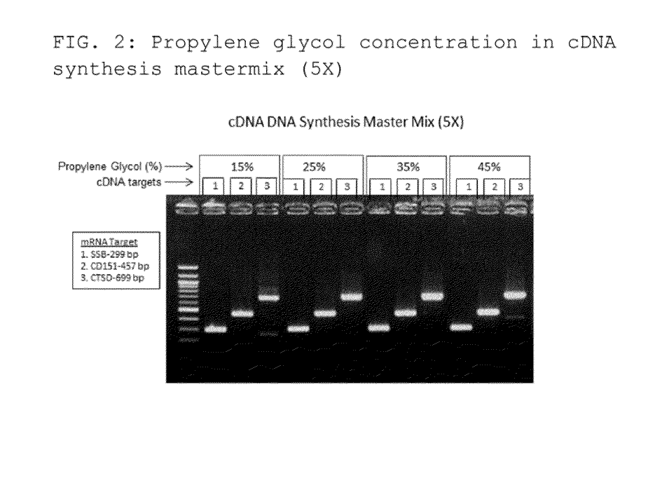

FIG. 1 shows stability information of cDNA synthesis mastermix. FIG. 2 show performance of various concentrations of propylene glycol mastermixes. Batch to batch reproducibility and reliability is shown in FIG. 3 and sensitive and robust section of various mRNA targets are demonstrated in FIG. 4 with these cDNA synthesis mastermixes. Propylene glycol based 5× cDNA synthesis mastermixes show high sensitivity (detect mRNA target from as low as 1 pg RNA, FIG. 4) and robust detection of various mRNA targets from 1-100 ng of total RNA. It is an anti-freeze format formulation storage as low as −35° C. and shown to be stable for more than 12 months when stored at −20° C.

Use of Primer Combinations

The present invention provides a more efficient and uniform priming for cDNA synthesis. The use of optimal concentration and combinations of random primers and oligo dT provides an efficient and representative conversion of mRNA sequences into cDNA regardless of distance from 3′ end of mRNA. The length of oligo dT can vary from 6 bases to 25 bases and 6 bases to nine base random primers (e.g., hexameric, heptameric, octameric, etc.). The amount of random primers can vary from 10 ng to 200 ng for each reaction (20 uL) and oligo dT to be used can be 2 nM to 50 nM.

Convenient, Stable, and Antifreeze Format Mastermix Compositions

Another embodiment is the form in which the reaction mixture is prepared and stably maintained. Traditionally, cDNA reaction components have been supplied as a number of separate components that are assembled into a complete reaction mix just prior to start of cDNA synthesis primarily due to storage stability issue. A typical kit for cDNA synthesis contains the following components: Oligo(dT) 12-18 (2.5 uM), Random hexamers (50 ng), RT Buffer (20 mM TrisCl pH 8.4, 50 mM KCl. d. 2.5 mM MgCl₂), 10 mM DTT, 0.5 mM dNTPs, 50 units MMLV RT, 40 units RNase inhibitor and stabilizer.

Each of the above components is provided separately and frozen at −20 degree for storage. The general belief has been that the components cannot be mixed for long term storage. A key component of these systems is reverse transcriptase that is always stored in special storage buffer with at least 50% glycerol, and is only added to the reaction mix immediately prior to start of cDNA synthesis.

Surprisingly, we have found that some or all of the components of the cDNA synthesis reaction can be combined and stored as a convenient ready-to-use mix that is stable to prolonged storage at −20 degree as an antifreeze format mastermix formulation and that can simply be added to a nucleic acid template solution when needed. The ready to use reaction mixture may contain between about 25% and about 50% propylene glycol to maintain stability of the RT enzyme that is present in the mix (FIG. 2).

Propylene glycol, also called propane-1,2-diol or propane-1,2-diol, is an organic compound with the chemical formula C₃H₈O₂. It is a viscous colorless, odorless and non-toxic liquid and able to lower the freezing point of water, and so it is used as antifreeze as well as aircraft de-icing fluid. It has been reported that the use of propylene glycol preserves viral RNA samples at room temperature for extended periods of time (Xianzhou Nie, et. al., Journal of Viological methods, Vol 175, pp224-227, 2011). It is also claimed that adding up 20% propylene glycol will enhance sensitivity and specificity in PCR amplification. (Ping-Hug Teng, et. al., US2012/0244599, 2012) A combination study of small amounts of propylene glycol (1.5 mM —less than 1%) and high concentrations of betaine (5.5M) has shown specificity improvement for high GC template DNA amplification (Fang Liu, et. al., Bioinformatics and Biomedical Engineering, 2009). However, there has been no prior publication reporting the use of propylene glycol as an essential stabilizing component in cDNA synthesis or RT-PCR amplification. We found that propylene glycol, unlike the case of glycerol, did not inhibit the RT activity even in the presence of high concentration propylene glycol and prevents from freezing of cDNA synthesis master even at −35 degrees storage.

Various format of cDNA synthesis mastermix can be successfully formulated (2× format to 6× format) for a variety of applications. The minimum components that may be usefully provided for the mixture are the propylene glycol, the RT and a suitable buffer component.

Suitable buffer compounds, such as Tris-HCl, HEPES, etc, are well known in the art. Metal ions necessary for RT activity, such as Mg and a monovalent cation such as K, Na, and NH₄ may be present in concentrations that are suitable for RT activity upon addition to a template solution. Additional components that may be present are a reducing agent, such as DTT, primer molecules such as gene specific primers, random primers of any suitable length, oligo(dT) compounds of any suitable length, anchored oligo(dT) molecules of suitable length, detergents or mixtures of detergents such as Tween, NP-40 and equivalent reagents, dNTPs, and one or more RNAse inhibitor proteins. The relative amounts contained in the mixture of such reagents necessary for use in RT reactions, when present, can be readily determined by a person of ordinary skill in the art. In addition, at least one thermostable DNA polymerase may also be present, which may be used for subsequent PCR reactions or the like.

Accordingly, the present invention provides newly improved, convenient, and ready to use configurations for cDNA synthesis. The methods of the invention reduce the number of additions for assembly of cDNA synthesis reactions which is highly sought by researchers especially in high throughput applications.

According to the methods of the invention, the ready to use mixes for cDNA synthesis can be made at different concentrations and provided as 2× to 6× “mastermixes”. The following is an example of a 5× mastermix for cDNA synthesis that contains all components necessary for cDNA synthesis according to the methods of this invention. Using 4 uL of this mastermix and RNA preparation of interest at a total volume of 20 uL provides a complete reaction mix for conversion of RNA into cDNA.

Formulation for 5× cDNA Synthesis Mastermix:

5× cDNA synthesis Mastermix: 250 mM Tris-HCl, pH 8.4, 375 mM KCl, 15 mM MgCl₂ 2.5 mM dNTP (each), 50 mM DTT, 2.5 nM oligo(dT)₂₀ 250 ng random primer, 40% propylene glycol, 0.1M trehalose, 0.005% NP-40, 150 units of MMLV RT, and 10 units RNase inhibitor protein (FIG. 1, FIG. 2, FIG. 3, and FIG. 4).

In addition to the above formulation, three other mastermixes were prepared that contained all reagents except the primers.

cDNA Synthesis Mastermix 1 did not have primers.

cDNA Synthesis Mastermix 2 contained oligo dT as the primers.

cDNA Synthesis Mastermix 3 contained random hexamers and octamers as primers.

All of the above 5× cDNA synthesis mastermixes were found to be stable for one year when stored at −20° C. (antifreeze format mastermix formulation storage as low as −35° C.). FIG. 1 shows the results and the efficacy of cDNA synthesis with repeated freeze-thaw cycle of these mastermixes.

It will be evident to those skilled in the art that a variety of different reverse transcriptases can be used according to the method of the invention. The reverse transcriptases may include, without limitation, AMV RT, RSV RT, MMLV RT, RNase H-mutants of various reverse transcriptases, HIV RT, EIAV RT, RAV2 RT, TTH DNA polymerase, C.hydrogenoformans DNA polymerase, SuperScript II RT, SuperScript RT, ThermoScript RT and mixtures thereof. It will also be obvious that one or more of the components of the above mastermix can be substituted with other equivalent reagent or protein. For example, there are a number of different RNase inhibitor proteins that can be used. Thermostable DNA polymerases suitable for use in the mastermixes are well known in the art and include Taq, Tth, Tne, Tma, Tli, Pfu, Pwo, Bst, Bca, Sac, Tac, Tfl, Tru, Mth, Mtb, and Mlep DNA polymerases and the like.

The composition of the 5× mastermix provided can also be varied, for example, by use of other buffers such as sulfate containing buffers or acetate based buffers that have been used for cDNA synthesis. It will be apparent to those skilled in the art that different formulations can be optimized for different applications (FIG. 4).

### Examples

FIG. 1: Freeze-Thaw Stability Test

Test sample (cDNA Synthesis Mastermix , 5× formulation) was subjected to number of freeze-thaw cycles as indicated. This 5× cDNA synthesis mastermix is an antifreeze format formulation storage as low as −35 degree so used ethanol-dry ice bath (−78 degrees) to test freeze thaw stability. Placed all test samples on ice and assembled reaction by adding 10 ng of total HeLa RNA (20 ul volume). cDNA synthesis reaction was carried out at 25 degree for 5 minutes then 30 minutes incubation at 45 degree, heat-killed RT upon incubation at 85 degree for 5 minutes and placed on ice. PCR reactions were assembled with 2× HS PCR Mix (LeGene). Added 2 ul of RT sample each and Ssb-299 primer (total 50 ul volume) and followed by 38 cycles of PCR amplification (94 degree, 15 s, 60 degree, 30 s, 68 degree, 1 min) and analyzed the amplified products by 1% agarose gel electrophoresis.

FIG. 2: Examination of Propylene Glycol Concentration (5×)

Prepared 15-45% of propylene glycol containing 5× cDNA mastermix as indicated. cDNA synthesis reaction (20 ul volume) was carried out at 25 degree for 5 minutes then 30 minutes incubation at 45 degree, heat-killed RT upon incubation at 85 degree for 5 minutes and placed on ice. PCR reactions were assembled with 2× HS PCR Mix (LeGene). Added 2 ul of RT sample each and Ssb-299, CD151-457, and CTSD-699 primers (total 50 ul volume) and followed by 38 cycles of PCR amplification (94 degree, 15 s, 60 degree, 30 s, 68 degree, 1 min) and analyzed the amplified products by 1% agarose gel electrophoresis.

FIG. 3: Reproducibility of cDNA Synthesis Mastermixes (5×). Test of 3 different lot samples were: (1). Lot A, (2). Lot B, and (3). Lot C cDNA Synthesis Mastermix (5× formulation). Reactions were assembled by adding 1 ng or 10 ng of total HeLa RNA as indicated (20 ul volume). cDNA synthesis reaction was carried out at 25 degree for 5 minutes then 30 minutes incubation at 45 degree, heat-killed RT upon incubation at 85 degree for 5 minutes and placed on ice. PCR reactions were assembled with 2× HS PCR Mix (LeGene). Added 2 ul of RT sample each and Ssb-299, Ptk-497, Hsm-603, and Gapdh-914 primers (total 50 ul volume) and followed by 38 cycles of PCR amplification (94 degree, 15 s, 60 degree, 30 s, 68 degree, 1 min) and analyzed the amplified products by 1% agarose gel electrophoresis.

FIG. 4: Performance of cDNA Synthesis Mastermixes (5×)

Test of 5 different mRNA targets were: (1). Gapdh-532 (1 pg, 10 pg, 100 pg total RNA input), (2). Ssb-299 (1 ng, 10 ng, 100 ng total RNA input), CBP-500 (1 ng, 10 ng, 100 ng total RNA input), Htb-1000 (1 ng, 10 ng, 100 ng total RNA input), Hrpa-1092 (1 ng, 10 ng, 100 ng total RNA input). Reactions were assembled by adding 1 ng or 10 ng of total HeLa RNA as indicated (total 20 ul volume). cDNA synthesis reaction was carried out at 25 degree for 5 minutes then 30 minutes incubation at 45 degree, heat-killed RT upon incubation at 85 degree for 5 minutes and placed on ice. PCR reactions were assembled with 2× HS PCR Mix (LeGene). Added 2 ul of RT sample each and Ssb-299, Ptk-497, Hsm-603, and Gapdh-914 primers (total 50 ul volume) and followed by 38 cycles of PCR amplification (94 degree, 15 s, 60 degree, 30 s, 68 degree, 1 min) and analyzed the amplified products by 1% agarose gel electrophoresis.

While the invention has been shown and described with reference to different embodiments thereof, it will be appreciated by those skilled in the art that variations in form, detail, compositions and operation may be made without departing from the spirit and scope of the invention as defined by the accompanying claims.

What is claimed is:

1. A reagent mixture comprising a ready to use reagent solution, wherein the solution comprises:
(a) propylene glycol in a concentration between about 25% and about 50%; and
(b) a viral reverse transcriptase in a concentration sufficient for use in a reverse transcription reaction without adding additional reverse transcriptase, wherein the viral reverse transcriptase is selected from the group consisting of Avian Myeloblastosis Virus Reverse Transcriptase, Respiratory Syncytial Virus Reverse Transcriptase, Moloney Murine Leukemia Virus Reverse Transcriptase, Human Immunodeficiency Virus Reverse Transcriptase, Equine Infectious Anemia Virus Reverse Transcriptase, Rous-Associated Virus 2 Reverse Transcriptase, Avian Sarcoma Leukosis Virus Reverse Transcriptase, RNaseH (−) Reverse Transcriptase, SuperScript II Reverse Transcriptase, and ThermoScript Reverse Transcriptase,
in a buffer suitable for use in a reverse transcription reaction, wherein the buffer comprises:
a co-factor metal ion necessary for reverse transcriptase activity; and
nucleoside triphosphates.

2. The mixture according to claim 1, wherein the buffer further comprises at least one primer suitable for priming reverse transcription of a template by the reverse transcriptase.

3. The mixture according to claim 1, wherein the buffer comprises an RNase inhibitor protein.

4. The mixture according to claim 1, wherein the buffer comprises a potassium salt, a magnesium salt, nucleoside triphosphates, Dithiothreitol, at least one primer suitable for priming reverse transcription of a template by the reverse transcriptase, at least one non-ionic detergent, and an RNase inhibitor protein.

5. The mixture according to claim 1, wherein the solution further comprises at least one viral reverse transcriptase enzyme.

6. The mixture according to claim 1, wherein the buffer further comprises at least one random primer.

7. The mixture according to claim 6, wherein the random primer is hexameric primer, heptameric primer, or octameric primer.

8. The mixture according to claim 1, wherein the buffer further comprises at least one oligo deoxy-thymine primer.

9. The mixture according to claim 8, wherein the oligo deoxythymine primer is deoxy-thymine 6-10 or deoxy-thymine 12-18.

10. The mixture according to claim 1, wherein the buffer further comprises at least one random primer and at least one oligo deoxy-thymine primer.

11. The mixture according to claim 1, wherein the co-factor metal ion necessary for reverse transcriptase activity is magnesium ion.

12. The mixture according to claim 1, wherein the buffer further comprises a monovalent cation.

13. The mixture according to claim 12, wherein the monovalent cation is K, Na, or NH₄.

14. The mixture according to claim 1, wherein the buffer further comprises a reducing agent.

15. The mixture according to claim 14, wherein the reducing agent is Dithiothreitol.

16. The mixture according to claim 1, wherein the buffer further comprises a non-ionic detergent.

17. The mixture according to claim 1, wherein the buffer further comprises trehalose.

18. The mixture according to claim 1, wherein the buffer further comprises glycerol in a concentration between about 2% and about 9%.

19. The mixture according to claim 1, wherein the solution is stable for 12 months to 2 years when stored at about −20° C.

20. The mixture according to claim 1, wherein the propylene glycol is in a concentration between about 30% and about 40%.
